(19) United States
(12) Patent Application Publication
(10) Pub. No.: US 2026/0027029 A1
(43) Pub. Date: Jan. 29, 2026

(54) ORAL CARE COMPOSITIONS FOR PROMOTING GUM HEALTH

(71) Applicant: The Procter & Gamble Company, Cincinnati, OH (US)

(72) Inventors: Ross STRAND, Singapore (CN); Sanjeev MIDHA, Mason, OH (US); Yunming SHI, Beijing (CN); Guannan WANG, Beijing (CN); Zhaoxin GAO, Beijing (CN)

(21) Appl. No.: 19/350,158

(22) Filed: Oct. 6, 2025

Related U.S. Application Data
(63) Continuation of application No. PCT/CN2023/086098, filed on Apr. 4, 2023.

(51) Int. Cl. A61K 8/46; A61K 8/04; A61K 8/21; A61K 8/24; A61K 8/29; A61K 8/34; A61K 8/44; A61K 8/73; A61K 8/81; A61Q 11/00

(52) U.S. Cl. CPC A61K 8/466; A61K 8/042; A61K 8/21; A61K 8/24; A61K 8/29; A61K 8/345; A61K 8/44; A61K 8/73; A61K 8/81; A61Q 11/00

(57) ABSTRACT

Oral care compositions comprising a stannous ion source, a surfactant combination of taurate and amphoteric surfactant (e.g., betaines), which are effective in treating dental plaque biofilm and neutralizing bacterial toxins, while providing an improved sensorial experience.

### FIELD OF THE INVENTION

The present invention relates to oral care compositions comprising stannous ion source and a taurate surfactant together with an amphoteric surfactant (e.g., betaines) for promoting oral Gum Health of a user. In particular, such oral care compositions are useful for controlling plaque bacteria and/or protecting from bacteria/toxin invasion.

### BACKGROUND OF THE INVENTION

Dental plaque (also known as dental biofilm) is a sticky, colorless deposit of bacteria that is constantly forming on the tooth surface. Dental plaque is generally made up of bacteria and extracellular polymer substances (so called “EPS”). EPS are biopolymers of microbial origin in which biofilm microorganisms are embedded. J. Bacteriol. 2007, 189 (22): 7945. Saliva, food and fluids combine to produce these deposits that collect where the teeth and gums meet. Plaque buildup is the primary factor in poor oral health that can lead to caries and periodontal (gum) disease, including gingivitis. One-way dentifrice compositions help prevent and control plaque is by leveraging anti-bacterial agents; however, the disadvantage and formulation challenge is the unintended reactivity of anti-bacterial agents with formulation ingredients. This may include oxidative degradation, hydrolysis, adsorption, or precipitation of oxy-hydroxide species, any of which can impact the bioavailability of the anti-bacterial agent.

Sodium lauryl sulfate (SLS) is widely used within dentifrice formulations, with broad benefits with broad compatibility with other ingredients, having a neutral taste and good foaming aesthetics. However, there is continuing consumer interest in developing oral care products that do not contain sodium lauryl sulfate. One of the concerns has been with the concerns that SLS can be a potential source for irritation, desquamation, and epithelial barrier dysfunction in the gingiva. Having a strong barrier function of the gingival epithelium is a physical barrier to separate the biofilm from the gingival tissue, providing the first line of defense against bacterial invasion in gingivitis or periodontal disease. Stannous fluoride has both, bactericidal and bacteriostatic effect on plaque bacteria. It also has been shown to decrease biofilm virulence of the bacterial endotoxins like lipopolysaccharide (LPS) and lipoteicoic acid (LTA) which are associated with the initialization of the inflammatory processes involved in periodontal disease and disruption of epithelial barrier function. Brushing with stannous fluoride toothpaste leads to reduction of gingivitis and as a result restoration of barrier function, what makes the gum tissue more resistance to bacteria attacks.

Thus, there is a continuing need to provide improved stannous and fluoride containing products for that help control plaque bacteria on teeth and/or protect from bacteria/toxin invasion, that do not contain sodium lauryl sulfate, but nevertheless have adequate stability, aesthetics and do not irritate or compromise the gingival epithelium.

### SUMMARY OF THE INVENTION

The present invention relates to an oral care composition comprising:
(a) from 0.01% to 5%, by weight of the composition, of a stannous ion source;
(b) from 0.1% to 5%, by weight of the composition, of a taurate surfactant represented by formula (I):

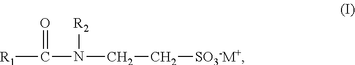

wherein R₁ is a saturated or unsaturated, straight, or branched alkyl chain with 6 to 18 C atoms; R₂ is H or methyl, and M is H, sodium, or potassium; and
(c) from 0.01% to 5%, by weight of the composition of an amphoteric surfactant.

Significant disruption and destabilization of dental biofilm can be achieved by an aqueous oral care composition comprising stannous ion source and a taurate surfactant together with amphoteric surfactant e.g., betaines. One advantage of the present invention is “better deep biofilm penetration and/or bacteria kill”. To this end, it is further surprisingly found that the penetration depth and/or penetration rate of stannous ion into the biofilms may be increased, when used in combination with Taurate. In short, the synergistic combination of taurate, betaines and stannous ion source in the oral care composition may be such that an improvement in the Gum Health benefit is achieved.

It is an advantage of the present invention to provide effective anti-bacterial properties by combining stannous with taurate surfactant and an amphoteric surfactant. It is a further advantage of the present invention to provide oral care compositions with improved deposition/penetration of Sn ions into the biofilm, as well as provide impact toxin neutralization of HGE healing recovery.

It is yet a further advantage that the oral care composition, is a dentifrice, and preferably provides pleasant sensorial experiences such as taste, foam, and mouth-feel experience.

It is yet a further advantage that the oral care composition exhibits good stability across a broad range of pH. It is yet a further advantage that the oral care compositions have physical and chemical stability across a range of manufacturing, handling and storage conditions. It is yet a further advantage that the oral care compositions have a stable quality of product (e.g., consistent visual appearance and no discoloration, etc.) even after three months storage at 40° C. It is yet a still further advantage that the oral care compositions of the present invention minimize the use of anti-bacterial agents.

It is yet a still further advantage that the oral care compositions of the present invention using surfactant combination of a Taurate and a betaine to reduce and/or eliminate the instability and/or discoloration problems of conventional stannous-containing toothpaste as described above. It is still a further advantage that the oral care composition of the present invention surprisingly provides improved sensorial profile and/or reduce potential astringency both during and after brushing.

The present invention further encompasses a method of treating dental biofilm comprising the step of brushing teeth with an oral care composition of the present invention.

The present invention further encompasses a method of preventing or mitigating plaque formation on tooth enamel comprising the step of brushing teeth with an oral care composition of the present invention.

### DETAILED DESCRIPTION OF THE INVENTION

### Definitions

As used herein, the articles including “a” and “an” when used in a claim, are understood to mean one or more of what is claimed or described.

The terms “alleviate”, and “alleviating” are used interchangeably and means minimizing, preventing, delaying, and/or treating at least one symptom of gum disease to effect positive change (i.e., benefit) to the user.

The term “biofilms” as used herein means a matrix-enclosed bacterial population adherent to each other and/or to surfaces or interfaces in the oral cavity.

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “oral care composition” or “oral care compositions” as used herein means a product that in the ordinary course of usage is retained in the oral cavity for a time sufficient to contact some or all the dental surfaces and/or oral tissues for purposes of oral activity. In one example, the composition provides a gum care benefit when used in the oral cavity. The oral care composition of the present invention may be in various forms including toothpaste, dentifrice, tooth gel, tooth powders, tablets, rinse, mouthwash, sub gingival gel, foam, mouse, chewing gum, lipstick, sponge, floss, prophy paste, petrolatum gel, denture adhesive, or denture product. In one example, the oral composition is in the form of a paste or gel. In another example, the oral composition is in the form of a dentifrice. The oral composition may also be incorporated onto strips or films for direct application or attachment to oral surfaces or incorporated into floss.

The term “dentifrice” as used herein means paste, gel, powder, tablets, or liquid formulations, unless otherwise specified, that are used to clean the surfaces of the oral cavity. Preferably the dentifrice compositions of the present invention are single phase compositions, although the compositions can be dual phase or multi-phase compositions. One example of a dentifrice composition is toothpaste (for brushing teeth). The term “teeth” as used herein refers to natural teeth as well as artificial teeth or dental prosthesis.

The term “partially soluble” or “partially water soluble” as used herein means a compound has a solubility of 1 g/1000 ml or more in water at 25° C. The term “insoluble” as used herein means a compound has a solubility less than 0.1 g/1000 ml at 25° C.

The term “effective amount” as used herein means an amount of a compound or composition sufficient to induce a positive benefit, an oral health benefit, and/or an amount low enough to avoid serious side effects, i.e., to provide a reasonable benefit to risk ratio, within the sound judgment of a skilled artisan. In one example, “effective amount” means at least 0.01% of the material, by weight of the composition, alternatively at least 0.1%.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “substantially free” as used herein refers to no intentional amount of that material is added to the composition or an amount of a material that is less than 0.05%, 0.01%, or 0.001% of the composition. The term “essentially free” as used herein means that the indicated material is not deliberately added to the composition, or preferably not present at analytically detectable levels. It is meant to include compositions whereby the indicated material is present only as an impurity of one of the other materials deliberately added. The term “free” as used herein refers to no reasonably detectable amount of that material is present in the composition.

The term “total water content” as used herein means both free water and water that is bound by other ingredients in the oral care composition.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt. %” herein. All molecular weights as used herein are weight average molecular weights expressed as grams/mole, unless otherwise specified.

All measurements referred to herein are made at 25° C. (i.e., room temperature) unless otherwise specified.

### Oral Care Compositions

It has been surprisingly discovered that the combination of stannous ion (i.e., an anti-bacterial agent) and an amino acid-based surfactant Taurate, together with an amphoteric surfactant, in an oral care composition is particularly useful for controlling plaque bacteria and maintaining integrity and such barrier function, i.e., promoting Gum Health benefits to users. In particular, the surprising discovery was that the penetration of the stannous ion into the biofilms is markedly improved when combined with the taurate and betaines. Without wishing to be bound by theory, the Taurate surfactant is derived from amino acid which contains both carboxylic and amine groups. It is believed that the stannous ions can bind strongly to these chemical moieties on amino acid to positively influence the penetration of stannous ions into the biofilms.

It has also been surprisingly found that the penetration depth and/or the penetration rate of stannous ions into the biofilms may be increased, or markedly increased, when formulated with Taurate surfactant, compared to other anionic surfactants like SLS. In short, the presence of taurate and betaine surfactants in combination with stannous ion source in the composition aids the composition's efficacy in mediating the harmful effects of the bacteria in the biofilms on the gums.

In one aspect, the present invention is directed to an oral care composition comprising:
(a) from 0.01% to 5%, by weight of the composition, of a stannous ion source;
(b) from 0.1% to 5%, by weight of the composition, of a taurate surfactant represented by formula (I)

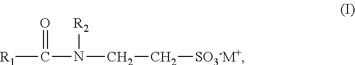

wherein R₁ is a saturated or unsaturated, straight, or branched alkyl chain with 6 to 18 carbon atoms; R₂ is H or methyl, and M is H, sodium, or potassium; and
(c) from 0.01% to 5%, by weight of the composition of an amphoteric surfactant.

### Stannous Ion Source

The present invention relates to the above-mentioned oral care compositions comprising, in a preferred example, the stannous ion source present in the amount of from 0.01% to 5%, preferably from 0.05% to 4%, or more preferably from 0.1% to 2%, by weight of the composition, to provide anti-bacterial effectiveness. The stannous ion source used herein may include any safe and effective stannous salt. Suitable examples of stannous ion source are selected from soluble stannous salts, and preferably selected from the group consisting of stannous chloride, stannous fluoride, stannous acetate, stannous gluconate, stannous oxalate, stannous sulfate, stannous lactate, stannous tartrate, stannous iodide, stannous chlorofluoride, stannous hexafluorozirconate, stannous citrate, stannous malate, stannous glycinate, stannous carbonate, stannous phosphate, stannous pyrophosphate, stannous metaphosphate, and combinations thereof; wherein preferably the stannous ion source is selected from stannous chloride, stannous fluoride, or a combination thereof.

In one preferred example, the stannous ion source comprises stannous chloride. In another preferred example, the stannous ion source comprises stannous fluoride. In a particular preferred example, the stannous ion source comprises a combination of stannous fluoride and stannous chloride.

In some examples, the oral care composition of the present invention is substantially free of insoluble stannous ion source stannous oxide, preferably essentially free of stannous oxide, more preferably free of stannous oxide.

### Taurate Surfactant

The oral care composition of the present invention contains a taurate surfactant as main surfactant. The Taurate is represented by formula (I):

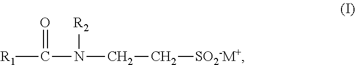

wherein R₁ is a saturated or unsaturated, straight, or branched alkyl chain with 6 to 18 C atoms; R₂ is H or methyl, and M is H, sodium, or potassium.

Preferably, the R₁ is a saturated or unsaturated, straight, or branched alkyl chain with 8 to 18 C atoms. Optionally but preferably, the taurate surfactant of the present invention comprises one or more selected from the group consisting of potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl Taurate, and combinations thereof.

### Secondary Surfactant

The oral care compositions herein may include a secondary surfactant, other than Taurate surfactant. Preferably, the secondary surfactant is an amphoteric surfactant, e.g., selected from the group consisting of betaine surfactants. The composition may include a secondary surfactant at a level of from about 0.01% to about 10%, from about 0.025% to about 9%, from about 0.05% to about 5%, from about 0.1% to about 2.5%, from about 0.5% to about 2%, or from about 0.1% to about 1%, by weight of the composition. Non-limiting examples of the amphoteric surfactant can be a betaine or a sultaine selected from the group consisting of: almondamidopropyl betaine, apricotamidopropyl betainc, avocadamidopropyl betaine, babassuamidopropyl betaine, bchenamidopropyl betaine, canolamidopropyl betaine, capryl/capramidopropyl betaine, cocoamidopropyl betaine, coco/olcamidopropyl betaine, coco/sunfloweramidopropyl betaine, cupuassuamidopropyl betaine, isostearamidopropyl betaine, lauramidopropyl betaine, meadowfoamamidopropyl betaine, milkamidopropyl betaine, minkamidopropyl r betaine, myristamidopropyl betaine, oatamidopropyl betaine, olcamidopropyl betaine, olivamidopropyl betaine, palmamidopropyl betaine, palmitamidopropyl betaine, palm kernelamidopropyl betaine, ricinolcamidopropyl betaine, sesamidopropyl betaine, shea butteramidopropyl betaine, soyamidopropyl betaine, stcaramidopropyl betaine, tallowamidopropyl betaine, undecylencamidopropyl betaine, wheat germamidopropyl betaine, cocamidopropyl hydroxysultaine (CAPHS), lauramidopropyl hydroxysultaine (LAPHS), olcamidopropyl hydroxysultainc (OAPHS), tallowamidopropyl hydroxysultaine (TAPHS), and combinations thereof; preferably selected from the group consisting of cocoamidopropyl betaine, lauramidopropyl betainc, olcamidopropyl betaine, tallowamidopropyl betaine, cocamidopropyl hydroxysultaine, and combinations thereof; and more preferably, selected from cocoamidopropyl betaine, lauramidopropyl betaine, or a combination thereof.

In one aspect, the composition of the present invention is substantially free of sodium lauryl sulfate (SLS). Preferably, the composition of the present invention is essentially free of, more preferably free of sodium lauryl sulfate (SLS). Without wishing to be bound by theory, such an approach may provide improvement on consumer's sensory feel by reducing irritation to oral tissue.

### Free of Insoluble Zinc Ion Source

Preferably the oral care composition of the present invention is substantially free of, preferably essentially free of, more preferably free of any insoluble or sparingly soluble zinc compounds. Insoluble or sparingly soluble zinc compounds, such as zinc oxide, or zinc phosphate, or zinc carbonate, is not suitable as the zinc source for the oral composition of the present invention.

In some embodiments, the oral care composition of the present invention may comprise from 0% to 2% of soluble or partially soluble zinc ion source such as zinc citrate, zinc chloride, zinc sulfate, zinc gluconate, zinc lactate, and combinations thereof. In some examples, the oral care composition comprises from 0.2% to 2% of soluble or partially soluble zinc ion source. in some other examples, the oral care composition is substantially free of any zinc ion source, preferably free of zinc ion source.

### Fluoride Ion Source

The compositions may optionally, but preferably, include an effective amount of an anti-caries agent, such as a fluoride ion source. The fluoride ion may be present in an amount sufficient to give a fluoride ion concentration in the composition at 25° C., and/or in one embodiment can be used at levels of from about 0.0025% to about 5% by weight of the composition, preferably from about 0.005% to about 2.0%, preferably from about 0.5% to about 1.5%, by weight of the composition, to provide anti-caries effectiveness. Representative fluoride ion sources include stannous fluoride, sodium fluoride, potassium fluoride, sodium monofluorophosphate, or mixtures thereof. In one aspect, the oral care compositions of the present invention may have a dual fluoride ion source, specifically sodium monofluorophosphate and an alkaline metal fluoride. Alternatively, the oral care compositions of the present invention may contain a dual fluoride ion source which is a combination of stannous fluoride and sodium fluoride. Without wishing to be bound by theory, such an approach may provide an improvement in mean fluoride uptake.

In some preferred examples, the fluoride ion source is selected from stannous fluoride, sodium monofluorophosphate, sodium fluoride, or combinations thereof.

In one aspect, the oral care composition of the present invention does not contain amine fluoride (also called olaflur) due to regulatory restraints in some regions.

### Calcium-Containing Abrasive

The compositions of the present invention can optionally, and in some aspects preferably, comprise from about 5% to about 50%, preferably from about 10% to about 50%, by weight of the composition, of a calcium-containing abrasive, wherein preferably the calcium-containing abrasive is selected from the group consisting of calcium carbonate, calcium glycerophosphate, dicalcium phosphate, tricalcium phosphate, calcium orthophosphate, calcium metaphosphate, calcium polyphosphate, calcium oxyapatite, sodium carbonate, and mixtures thereof; wherein more preferably the calcium-containing abrasive is calcium carbonate. Preferably, the composition comprises from about 15% to about 50%, preferably from about 15% to about 40%, preferably from about 15% to about 25%, by weight of the composition, of a calcium-containing abrasive. Preferably, the calcium-containing abrasive is calcium carbonate. More preferably, the calcium-containing abrasive is selected from the group consisting of fine ground natural chalk, ground calcium carbonate, precipitated calcium carbonate, and combinations thereof. Non-limiting examples of the weight percentages of the calcium-containing abrasive include: about 15%, about 20%, about 25%, or about 30%, by weight of the composition, preferably wherein the calcium-containing abrasive is calcium carbonate.

### Silica Abrasive

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. For example, the silica abrasives used in the present invention is a precipitated silica (e.g., sodium silicate solution by destabilizing with acid as to yield very fine particles) such as those from the ZEODENT® series from Huber Engineered Materials (e.g., ZEODENT® 103, 124, 113 115, 163, 165, 167). It is acknowledged that some of these silicas (e.g., synthetic amorphous silica) can perform both abrasive and thickening functions but are included herein under the term “abrasive” for purposes of the present invention. Preferably the oral care composition comprises from 1% to 35%, more preferably from 5% to 25% of abrasive, by weight of the composition.

pH

The pH of the oral care composition may be ranging from about 4.0 to about 9.0, preferably from about 4.3 to about 8.0, more preferably from about 4.6 to about 7.5. In some examples, the composition has a pH ranging from 4.0 to 4.9. In alternative examples, the composition may have a pH ranging from 5.0 to 6.0. In other examples, the composition has a pH ranging from 6.1 to 7.3. In some other examples, the composition may have a pH ranging from 7.5 to 9.0. A method for assessing pH of oral care is described is below. For purposes of clarification, although the analytical method describes testing the oral care composition when freshly prepared, for purposes of claiming the present invention, the pH may be taken at any time during the product's reasonable lifecycle (including but not limited to the time the product is purchased from a store and brought to the user's home).

### pH Modifying Agent

The oral care compositions herein may include an effective amount of a pH modifying agent, preferably wherein the pH modifying agent is a pH buffering agent. The pH modifying agents, as used herein, refer to agents that can be used to adjust the pH of the oral care compositions to the above-identified pH range. The compositions can comprise from about 0.001% to about 5%, by weight of the composition, of pH modifying agent. The pH modifying agents may include alkali metal hydroxides, ammonium hydroxide, organic ammonium compounds, carbonates, sesquicarbonates, borates, silicates, phosphates, imidazole, and mixtures thereof. Specific pH agents include monosodium phosphate (monobasic sodium phosphate or “MSP”), trisodium phosphate (sodium phosphate tribasic dodecahydrate or “TSP”), sodium benzoate, benzoic acid, sodium hydroxide, potassium hydroxide, alkali metal carbonate salts, sodium carbonate, imidazole, pyrophosphate salts, tripolyphoshpate salts, sodium gluconate, lactic acid, sodium lactate, citric acid, sodium citrate, phosphoric acid. Without wishing to be bound by theory, phosphate may also have calcium ion chelating activity and therefore provide some monofluorophosphate stabilization (in those formulations containing monofluorophosphate).

A method for assessing pH of oral care is described. The pH is measured by a pH Meter with Automatic Temperature Compensating (ATC) probe. The PH Meter is capable of reading to 0.001 pH unit. The pH electrode may be selected from one of the following (i) Orion Ross Sure-Flow combination: Glass body-VWR #34104-834/Orion #8172BN or VWR #10010-772/Orion #8172BNWP; Epoxy body-VWR #34104-830/Orion #8165BN or VWR #10010-770/Orion #8165BNWP; Semi-micro, epoxy body-VWR #34104-837/Orion #8175BN or VWR #10010-774/Orion #3175BNWP; or (ii) Orion PerpHect combination: VWR #34104-843/Orion #8203BN semi-micro, glass body; or (iii) suitable equivalent. The automatic temperature compensating probe is Fisher Scientific, Cat #13-620-16.

A 25% by weight slurry of oral care is prepared with deionized water, and thereafter is centrifuged for 10 minutes at 15000 rotations-per-minute using a SORVALL RC 28S centrifuge and SS-34 rotor (or equivalent gravitational force, at 24149 g force). The pH is assessed in supernatant after one minute or the taking reading is stabilized. After each pH assessment, the electrode is washed with deionized water. Any excess water is wiped with a laboratory grade tissue. When not in issue, the electrode is kept immersed in a pH 7 buffer solution or an appropriate electrode storage solution.

### Humectants

The oral care compositions herein may include humectants present in the amount of from 0% to 70%, or from 15% to 55%, by weight of the compositions. Humectants keep oral care compositions from hardening upon exposure to air and certain humectants may also impart desirable sweetness of flavor to oral care compositions. Suitable examples of humectants may include glycerin, sorbitol, polyethylene glycol, propylene glycol, xylitol, trimethyl glycine, and mixtures thereof. Other examples may include other edible polyhydric alcohols. In some examples, the humectant is selected from sorbitol, glycerin, and combinations thereof. In a preferred example, the humectant is sorbitol. In another preferred example, the humectant is glycerin. In an example, the composition comprises from 10% to 66%, alternatively from 20% to 55%, of humectant by weight of the composition.

### Water

The oral care compositions herein may include from 10% to 60%, by weight of the composition, of total water content. In some examples, the oral care composition may include from 10% to 50%, or preferably from 10% to 40%, by weight of the composition, of total water content. The term “total water content” as used herein means the total amount of water present in the composition, whether added separately or as a solvent or carrier for other raw materials but excluding that which may be present as water of crystallization in certain inorganic salts. Preferably, the water is USP water.

Water is commonly used as a carrier material in oral care compositions due to its many benefits. For example, water is useful as a processing aid, is benign to the oral cavity and assists in quick foaming of toothpastes. Water may be added as an ingredient in its own right or it may be present as a carrier in other common raw materials such as, for example, sorbitol.

### Thickening System

The oral care compositions of the present invention may comprise a thickening system. Preferably the oral care composition comprises from about 0.5% to about 10%, preferably from about 0.8% to about 5%, more preferably from about 1% to about 5%, by weight of the composition, of the thickening system. More preferably the thickening system comprises a thickening polymer, a thickening silica, or mixtures thereof. Yet more preferably, when the thickening system comprises a thickening polymer, the thickening polymer is selected from a carboxymethyl cellulose, a linear sulfated polysaccharide, a natural gum, or mixtures thereof. Yet still more preferably, when the thickening system comprises a thickening polymer, the thickening polymer is selected from the group consisting of: (a) from about 0.01% to about 3% of a carboxymethyl cellulose (“CMC”) by weight of the composition, preferably from about 0.1% to about 2.5%, more preferably from about 0.5% to about 1.7%, by weight of the composition, of CMC; (b) from about 0.01% to about 2.5%, preferably from about 0.05% to about 2%, more preferably from about 0.1% to about 1.5%, by weight of the composition, of a linear sulfated polysaccharide, preferably wherein the linear sulfated polysaccharide is a carrageenan; (c) from about 0.01% to about 3%, preferably from about 0.1% to about 2%, more preferably from about 0.2% to about 1.8%, by weight of the composition, of a natural gum; or (d) mixtures thereof.

Thickening polymer can also include either a non-colloidal microcrystalline cellulose, colloidal microcrystalline cellulose, or a mixture thereof. Non-colloidal microcrystalline cellulose, typically called microcrystalline cellulose or MCC, is a purified, partially depolymerized cellulose, e.g., AVICEL® PH 102 or PH 105. Colloidal microcrystalline cellulose is obtained by reducing the particle size of microcrystalline cellulose and stabilizing the particles to avoid formation of hard aggregates, e.g., AVICEL® CL 611 or RC 591.

Preferably, when the thickening system comprises a thickening silica, the thickening silica is from about 0.01% to about 8%, preferably from about 0.1% to about 5%, preferably about 1% to about 3%, by weight of the composition.

Preferably, the linear sulfated polysaccharide is a carrageenan (also known as carrageenan). Examples of carrageenan include Kappa-carrageenan, Iota-carrageenan, Lambda-carrageenan, or mixtures thereof.

In one aspect the thickening silica is obtained from sodium silicate solution by destabilizing with acid as to yield very fine particles. One commercially available example is ZEODENT® branded silicas from Huber Engineered Materials (e.g., ZEODENT® 103, 124, 113 115, 163, 165, 167).

In one aspect, the CMC is prepared from cellulose by treatment with alkali and monochloro-acetic acid or its sodium salt. Different varieties are commercially characterized by viscosity. One commercially available example is Aqualon™ branded CMC from Ashland Special Ingredients (e.g., Aqualon™ 7H3SF; Aqualon™ 9M3SF Aqualon™ TM9A; Aqualon™ TM12A).

Preferably, a natural gum is selected from the group consisting of gum karaya, gum arabic (also known as acacia gum), gum tragacanth, xanthan gum, and mixtures thereof. More preferably the natural gum is xanthan gum. Xanthan gum is a polysaccharide secreted by the bacterium Xanthomonas camestris. Generally, xanthan gum is composed of a pentasaccharide repeat units, comprising glucose, mannose, and glucuronic acid in a molar ratio of 2:2:1, respectively. The chemical formula (of the monomer) is C₃₅H₄₉O₂₉. In one aspect, the xanthan gum is from CP Kelco Inc (Okmulgee, US).

In one aspect, the thickening polymer is selected from group consisting of a carboxymethyl cellulose, a linear sulfated polysaccharide, a natural gum, and mixtures thereof.

### Viscosity

Preferably the oral care compositions of the present invention have a viscosity range from about 200,000 centipoises to about 850,000 centipoises (“cP”). A method for assessing viscosity is described. The viscometer is Brookfield® viscometer, Model DV-I Prime with a Brookfield “Helipath” stand. The viscometer is placed on the Helipath stand and leveled via spirit levels. The E spindle is attached, and the viscometer is set to 2.5 RPM. Detach the spindle, zero the viscometer and install the E spindle. Then, lower the spindle until the crosspiece is partially submerged in the paste before starting the measurement. Simultaneously turn on the power switch on the viscometer and the helipath to start rotation of the spindle downward. Set a timer for 48 seconds and turn the timer on at the same time as the motor and helipath. Take a reading after the 48 seconds. The reading is in cP.

### Colorant

The compositions herein may include a colorant. Titanium dioxide is one example of a colorant. Titanium dioxide is a white powder which adds opacity to the compositions. Colorant, e.g., titanium dioxide, generally can comprise from about 0.25% to about 5%, by weight of the composition.

### Flavoring Agent

The dentifrice composition herein may include from 0.01% to 5%, preferably from 0.1% to 2%, by weight of the composition, of a flavoring agent. Examples of suitable flavoring agent that may be used in the dentifrice composition include those described in U.S. Pat. No. 8,691,190; Haught, J. C., from column 7, line 61 to column 8, line 21. In some examples, the flavoring agent may be selected from methyl salicylcate, menthol, eugenol, and cineol. In some examples, the dentifrice composition may comprise a flavor mixture which is free of or substantially free of methyl salicylcate, menthol, eugenol, and cineol.

### Sweetener

The dentifrice compositions herein may include a sweetening agent. The sweetening agent is generally present in the dentifrice compositions at levels of from 0.005% to 5%, by weight of the composition. Suitable examples of sweetener include saccharin, dextrose, sucrose, lactose, xylitol, maltose, levulose, aspartame, sodium cyclamate, D-tryptophan, dihydrochalcones, acesulfame, sucralose, neotame, and mixtures thereof. Other suitable examples of sweetener are described in U.S. Pat. No. 8,691,190; Haught, J. C. from column 9, line 18 to column 10, line 18.

### Other Ingredients

The present oral care composition can comprise the usual and conventional ancillary components that are known to one skilled in the art. Optional ingredients include, for example, but are not limited to, anti-plaque agent, anti-sensitivity agent, whitening and oxidizing agent, anti-inflammatory agent, anti-calculus agent, chelating agent, tooth substantive agent, analgesic, and anesthetic agent. It will be appreciated that selected components for the oral care compositions must be chemically and physically compatible with one another.

Preferably, the oral care composition of the present invention is essentially free of glycyrrhetinic acid and salt thereof.

### Method of Use

In one aspect, the present invention relates to a method for treating dental biofilm. the method comprises the step of brushing/cleaning teeth with an oral care composition according to the present invention. The method comprises contacting a subject's teeth with the oral care compositions according to the present invention.

In another aspect, the present invention also relates to a method of controlling plaque bacteria in a subject comprising administering to the subject's oral cavity an oral care composition according to the present invention, wherein preferably the administering occurs at least once a day, more preferably at least twice a day.

### Test Methods

### Test Method 1: Barrier Protection Test

Barrier Protection Test is described here. This test assesses the barrier protection ability of a leave-on oral care composition by using capsaicin to treat a cell covered by the sample.

### Cell Culture:

Human gingival epithelial (HGE) cell is maintained in a 37° C. incubator with 5% CO2 saturation. Adding 10% wt. fetal calf serum, and 1% wt. penicillin-streptomycin to Dulbecco's Modified Eagle's Medium (DMEM, Gibco) to make the complete culture medium for these cells, which is changed every 2-3 days.

### Barrier Protection Analysis by Real Time Cell Analyzer (RTCA):

The real time cell analyzer (RTCA) xCELLigence RTCA DP (ACEA Bioscience Inc., San Diego, CA) equipment, based on impedance measurement, is used to monitor the barrier protection of sample. 50 μl of the complete culture medium is added to E-plates to obtain background readings, followed by the addition of 100 ul of the cell suspension. The E-plates containing 28000 cell/well are incubated at 37° C., 5% CO2 for 22-24 h until cells are confluent. The culture medium is changed to DMEM or DMEM with 50 times diluted sample (sample information is as Table 2 below) and is incubated at 37° C., 5% CO2 for 2 h. The barrier disruptor (capsaicin to a final concentration of 75 uM in this case) is added to wells of the E-plate. Impedance is reflected as a Cell Index (“CI”) and recorded continuously for 16-24 h and reported as a CI curve.

The CI curve is normalized at the time point just before adding samples. The normalized CI at 3 h, after adding the barrier disruptor, is compared to blank control are recorded. The barrier protection percentage is calculated by the below formulation:

Barrier Protection Percentage=(normalized CI of sample−normalized CI of capsaicin)/normalized CI of blank.

### Test Method 2: Assay for Measuring Improve Endotoxin Neutralization of Anti-Bacterial Agent in the Biofilms

To determine improved endotoxin neutralization of anti-bacterial agent in the biofilms, the following assay is used to assess LPS binding efficiency of stannous ions via measurement of a fluorescent dye that is bound to lipid A of LPS in in situ plaque biofilms for inventive oral care compositions of the present invention and controls. Details of the assay are described below.

### 1. Substrate for Biofilm Growth

Hydroxyapatite (“HA”) disks are used for in situ growth of biofilms. The HA disks are designed having three parallel grooves (i.e., 200 μm wide; 200 μm deep for two sides' grooves; while 500 μm wide and 500 μm deep for the middle groove) in each disk. When attaching disks to subject's mouth, keep these grooves vertical, to mimic interproximal gap between teeth, which is the hard-to-clean area where plaque generally tends to accumulate. This model allows the collection of undisturbed plaque from the grooves. HA disks are manufactured by Shanghai Beierkang biomedicine limited company.

### 2. Wearing the Splint

Human subjects wear the splint. Each subject wears up to 12 HA disks on the splint to ensure that, at least, 9 HA disks are available after 48 hours. In a specific example, and with reference to FIG. 2, the HA disk has three parallel grooves (the two sides' grooves are 300 μm wide and 300 μm deep; while the middle grove (in between the two side grooves) is 500 μm wide and 500 μm deep). The middle groove is designed wider and deeper than the two sides' grooves so that the HA disk can be more easily separated into two identical half-disks for head-to-head comparison purposes. Further details of the HA disks are described in US2017/0056531 (e.g., paragraphs [0019]-[0020]).

The disks can be positioned such that the recede is in the inter-dental space between the teeth (since this location is prone to plaque (given the difficulty in cleaning, etc.)). The subjects withdraw the splint only during meals (the splint stored in an opaque container in humid conditions) and to perform oral hygiene procedures. Immediately thereafter, the splint is worn again. Subjects are asked to use a straw when drinking.

3. In-Situ Biofilms Release from HA Desk

All HA disks are removed from the splint at 48 hours by tweezers. Tweezers are used to hold the edge of HA chips and transfer the HA disk to a 2 mL centrifuge tube containing phosphate buffered saline (PBS) solution. Tweezers are washed thoroughly (water; 75% alcohol; and then deionized water) before every disk transfer.

### 4. Preparation of Toothpaste Supernatant

15 grams of deionized water is added to 5 grams toothpaste of testing samples. After stirring thoroughly, the mixture is centrifuge at 12,000 RPM for 20 minutes. The supernatant is prepared one day before usage and stored at 4° C.

### 5. Confocal Laser Scanning Microscopy

After the HA disks are removed from the splint. The HA disks are used for ex vivo treatment by the different inventive and control compositions. After being treated with the subject supernatant and labeled with microbial fluorescent probe and stannous fluorescent probe, the biofilms in the grooves are measured by confocal laser scanning microscopy (“CLSM”) (as described below). Preferably, the neutralized-LPS fluorescent probe is BODIPY-TR-cadaverine (BC) (available from Thermo Fisher). Preferably, the microbial fluorescent probe is the Molecular Probes™ LIVE/DEAD® BacLight™ system (available from Thermo Fisher).

### 6. Disk Preparation

The HA disks are rinsed in PBS solution and each HA disk is divided into two halves by tweezers. Thereafter, each half-disk is placed into 500-1000 μL of PBS solution statically for 1 minute. Each disk is treated for two minutes by either PBS solution or toothpaste supernatant. Each disk is washed by holding each disk with tweezers, shaken for ten rounds of back and forth in 1 mL of PBS solution, and then this washing cycle is repeated. Then each disk is immersed into 500-1000 μL PBS solution statically for 5 minutes.

### 7. Fluorescence Staining and Microscopy

The LPS neutralization effect is evaluated using BODIPY-TR-cadaverine (BC), a fluorescent dye that is bound to lipid A, thereby suppressing its fluorescence. BC is displaced by agents with an affinity for this lipid. When LPS are bound by other ions, e.g., stannous, BC is released from the LPS, and its fluorescence is proportional to the amount of free (unbound) BC present. Therefore, the level of fluorescence indicates the amount of neutralized (bound) LPS versus free (unbound) LPS, and the efficacy of an antibacterial agent in reducing the biofilm's toxicity. The greater the amount of bound LPS, the lower its toxicity.

After treatment and immersing, each half-disk is stained with the BODIPY-TR-cadaverine (BC) probe together with Syto-9 probe (containing 5 μM BC probe and 5 μM Sn probe) for 30 minutes in the dark. After staining, each disk is immersed into 500-1000 μL PBS solution statically for 2 minutes. The disks are washed again, by holding each disk with tweezers, shaken for five rounds of back and forth in 1 mL PBS solution, and repeated. For SYTO-9/BC dye-stained samples, the following parameters are used: λex=488 nm/543 nm, λem=500/580 nm, 20× objective lens, and scanning from bottom of surface bacteria for 60 μm with step size=3 μm.

### 8. Confocal Laser Scanning Microscopy

The Leica™ TCS SP8 AOBS spectral confocal microscope is used. The confocal system consists of a Leica™ DM6000B upright microscope and a Leica™ DMIRE2 inverted microscope. An upright stand is used for applications involving slide-mounted specimens, whereas the inverted stand, having a 37° C. incubation chamber and CO2 enrichment accessories, provides for live cell applications. The microscopes share an exchangeable laser scan head and, in addition to their own electromotor-driven stages, a galvanometer-driven high precision Z-stage which facilitates rapid imaging in the focal (Z) plane. In addition to epifluorescence, the microscopes support a variety of transmitted light contrast methods including bright field, polarizing light and differential interference contrast, and are equipped with 5×, 20×, 40×, 63× (oil and dry) and 100× (oil) Leica™ objective lenses.

The laser scanning and detection system is described. The TCS SP8 AOBS confocal system is supplied with four lasers (one diode, one argon, and two helium neon lasers) thus allowing excitation of a broad range of fluorochromes within the UV, visible and far-red ranges of the electromagnetic spectrum. The design of the laser scan head, which incorporates acousto-optical tunable filters (“AOTF”), an acousto-optical beam splitter (“AOBS”) and four prism spectrophotometer detectors, permits simultaneous excitation and detection of three fluorochromes. The upright microscope also has a transmission light detector making it possible to overlay a transmitted light image upon a fluorescence recording.

Leica™ Confocal software is used. The confocal is controlled via a standard Pentium PC equipped with dual monitors and running Leica™ Confocal Software. The Leica Confocal Software provides an interface for multi-dimensional image series acquisition, processing, and analysis, that includes 3D reconstruction and measurement, physiological recording, and analysis, time-lapse, fluorochrome co-localization, photo-bleaching techniques such as FRAP and FRET, spectral mixing and multicolour restoration. Regarding image analysis, the SYTO-9/BC probe-stained samples are chosen to quantify fluorescence intensity of red and green pixels.

### LPS Endotoxin Neutralization Efficacy

Using the software, the fluorescence intensity ratio (FIR) of bound LPS/bacterial cell is calculated. This ratio of fluorescence intensity indicates the relative amount of bound (neutralized) LPS per unit of bacteria, and the efficacy of an agent in reducing the biofilm's toxicity. The greater the fluorescence intensity ratio, the higher LPS endotoxin neutralization efficacy.

### Active Penetration Rate in Biofilm

Using the software, the pixel overlaps of “green” bacterial probes and that of “red” stannous probes are identified, and then this value is divided by all non-black pixels (that include non-overlapping stannous probes) to provide a co-localization percentage of stannous in bacteria. Generally, the higher this co-localization percentage, the more efficacious the oral care product is in delivering stannous into bacteria. (See Xiang J, Li H, Pan B, Chang J, He Y, He T, Strand R, Shi Y, Dong W. (2018) Penetration and Bactericidal Efficacy of Two Oral Care Products in an Oral Biofilm Model. Am J Dent, Vol. 31, Issue 1:53-60)

### Test Method 3: RCTA Test—2D-Cell Based Method for Cell Barrier Disruption

Cellular barrier stability is monitored by a non-invasive, label-free manner by continuous impedance measurement using the microelectronic biosensor system for cell-based assays xCELLigence®. The continuous monitoring quantifies the cell health, proliferation, morphology change and barrier function real time kinetic assay.

### (1) Cell Culture:

Human gingival epithelial (HGE) cell is maintained in a 37° C. incubator with 5% CO2 saturation. Dulbecco's modified Eagle's medium (DMEM, Gibco) containing 10% fetal calf serum, 1% penicillin-streptomycin is used as the complete culture medium, which is changed every 2-3 days.

### (2) Toothpaste Preparation:

6+/−0.001 g toothpaste is added to 18 ml DMEM in 100 ml sterilized cup. The sample is stirred at 500 rpm for 20 min using magnetic stir bar. The slurry is then transferred to a 50 ml sterilized tube and centrifuged at 15000 rpm for 15 min. The supernatant is collected and further diluted by DMEM to 0.05%.

### (3) Barrier Function Analysis by Real Time Cell Analyzer (RTCA):

The real time cell analyzer (RTCA) xCELLigence RTCA DP (ACEA Bioscience Inc., San Diego, CA) equipment, based on impedance measurement, is used to monitor the barrier disruption of the samples. 50 ul complete culture medium is added to E-plates to obtain background readings, followed by the addition of 100 ul of the cell suspension. The E-plates containing 40000 cell/well are incubated at 37° C., 5% CO2 for 22-24 h until cells are confluent to form a monolayer barrier. The culture medium is changed to DMEM or DMEM with 0.05% of prototypes and incubate at 37° C., 5% CO2 for 2 h. Impedance as reflected by the Cell Index (CI) is recorded continuously.

### (4) Cellular Impedance Evaluation:

The CI curve is normalized at the time point just before addition of the sample prototypes. The normalized CI at 2 h after adding prototypes is compared to blank control to generate the percentage of barrier function for comparison and analysis. The statistical analysis is by the JMP student's t-test.

### EXAMPLES

The following examples and descriptions further clarify embodiments within the scope of the present invention. These examples are given solely for the purpose of illustration and are not to be construed as limitations of the present invention as many variations thereof are possible without departing from the spirit and scope.

### Example A: Preparation of Examples 1 to 5

Examples 1 to 5 are dentifrice compositions shown below with amounts of components in wt %. Examples 1 and 2 are comparative stannous free formulations containing sodium lauryl sulfate surfactant (SLS) and taurate surfactant respectively. Examples 3 and 4 are stannous containing formulations with sodium lauryl sulfate surfactant (SLS) and taurate surfactant respectively. Example 5 is an inventive formulation according to the present invention, which contains stannous ion source, a taurate surfactant (SMCT), and an amphoteric surfactant (cocamidopropyl betaine). All the compositions are prepared by admixture of the components in Table 1, in the proportions indicated. A commercial comparative formulation which contains insoluble zinc ion and stannous ion source is prepared, namely Example 6. Additionally, a commercial comparative formulation which contains insoluble zinc ion, soluble zinc ion and a surfactant combination of a taurate and cocamidopropyl betaine is prepared as well, namely Example 7.

TABLE 1
Examples 1 to 5

| Ingredients | Ex. 1 | Ex. 2 | Ex. 3 | Ex. 4 | Inventive Ex. 5 |
|---|---|---|---|---|---|
| Water | Q.S. | Q.S. | Q.S. | Q.S. | Q.S. |
| Glycerin | — | — | 27.000 | 27.000 | 27.000 |
| Sorbitol (70%) | 65.200 | 65.200 | 35.000 | 35.000 | 35.000 |
| Stannous Fluoride | — | — | 0.454 | 0.454 | 0.454 |
| Stannous Chloride & Silica (10%) | — | — | 0.562 | 0.562 | 0.562 |
| Sodium Fluoride | 0.321 | 0.321 | — | — | — |
| Sodium Gluconate | — | — | 1.150 | 1.150 | 1.150 |
| Sodium Hydroxide (50%) | — | — | 0.740 | 0.740 | 0.740 |
| MonoSodium Phosphate | 0.400 | 0.400 | — | — | — |
| TriSodium Phosphate | 0.930 | 0.930 | — | — | — |
| Sodium Saccharin | 0.300 | 0.300 | 0.500 | 0.500 | 0.500 |
| Xathan Gum | — | — | 0.613 | 0.613 | 0.613 |
| Carrageenan | — | — | 1.050 | 1.050 | 1.050 |
| Carbomer | 0.220 | 0.220 | — | — | — |
| Sodium Carboxymethyl Cellulose | 0.700 | 0.700 | — | — | — |
| CMC | | | | | |
| Sodium Citrate | — | — | 1.550 | 1.550 | 1.550 |
| Zinc Citrate | — | — | — | — | — |
| Titanium Dioxide | — | — | — | — | — |
| Hydrated Silica | 16.000 | 16.000 | 15.000 | 15.000 | 15.000 |
| Sodium Lauryl Sulfate (29%) | 5.000 | — | 5.000 | — | — |
| Sodium Methyl Cocoyl Taurate | — | 5.000 | — | 5.000 | 5.000 |
| (29%) | | | | | |
| Cocoamidopropyl Betaine | — | — | — | — | 2.000 |
| Flavor | 1.000 | 1.000 | 1.000 | 1.000 | 1.000 |
| Total | 100.000 | 100.000 | 100.000 | 100.00 | 100.00 |

### Example B: Improve Endotoxin Neutralization & Stannons (Sn) Penetration in the Biofilm

Test method 2 is conducted to measure the bacterial Endotoxin neutralization in Biofilm of the Inventive and comparative examples, as well as to measure the active Sn penetration rate in biofilm that neutralizes the endotoxin. The results are provided in Table 2 and Table 3.

TABLE 2
Bacterial Endotoxin neutralization in biofilm

| | Fluorescence intensity | |
|---|---|---|
| | ratio (FIR) Bound LPS/ | |
| Treatment | Bacterial Cell | Grouping * |
| Negative Control (Phosphate | 0.1033 | E |
| Buffer solution) | | |
| Example 1 | 0.1267 | E |
| Example 2 | 0.1300 | E |
| Example 3 | 1.0600 | C |
| Example 4 | 1.3767 | B |
| Example 5 | 2.1400 | A |
| ¹Example 6 | 0.8500 | D |
| ²Example 7 | 0.1467 | E |

* means for groups in homogeneous subsets, those that do not share a letter are significantly different (alpha = 0.05), and mean sample size = 6. Student-Newman-Keuls method
¹Comparative Example 5 Colgate ® Total TM SF Toothpaste (LOT 1249US56C1), having the ingredients: Water, Sorbitol, Hydrated Silica, Glycerin, PEG-12, Tetrasodium Pyrophosphate, Flavor, Sodium Lauryl Sulfate, Zinc Phosphate, Cellulose Gum, Sodium Citrate, Stannous Fluoride, Microcrystalline Cellulose, Sodium Saccharin, cocamidopropyl Betaine, Xanthan Gum, Citric Acid, Sucralose, Titanium Dioxide.
²Comparative Example 6 Colgate ® Miracle Repair Toothpaste (2014CN59 EXP20250114), having the ingredients: glycerin, water, hydrated silica, Sodium Methyl Cocoyl Taurate, flavor, arginine, zinc oxide, cocamidopropyl betaine, cellulose gum, zinc citrate, tetrasodium pyrophosphate, xanthan gum, benzyl alcohol, sodium saccharine, sodium fluoride, phosphoric acid

TABLE 3
Active Sn penetration rate in biofilm

| Treatment | Sn Penetration % | Grouping * |
|---|---|---|
| Negative Control (Phosphate | 0.1067 | E |
| Buffer solution) | | |
| Example 1 | 1.5333 | E |
| Example 2 | 1.6667 | E |
| Example 3 | 75.907 | C |
| Example 4 | 90.913 | B |
| Example 5 | 95.673 | A |
| ¹Example 6 | 64.001 | D |
| ²Example 7 | 1.9700 | E |

* Same as above.
¹Same as above.
²Same as above.

The results in Table 2 demonstrate the significantly higher Fluorescence Intensity Ratio (FIR) of bound LPS/bacterial cell with the biofilms treated with the Examples 4 (i.e., Sn+Taurate containing compositions) over both the Comparative Example 3 (i.e., Sn+SLS compositions), and the Example 6 (containing Sn, SLS and co-surfactant cocoamidopropyl betaine). More surprisingly, the Inventive Example 5 (containing Sn+Taurate+Cocoamidopropyl betaine) has greater bacterial toxin neutralization than that of Example 4 of Sn+Taurate only. Further those Comparative Examples 1 and 2 (i.e., containing no Sn source) have no effect on the endotoxin neutralization irrespective of the surfactant type taurate or SLS.

The results in Table 3 demonstrate significantly higher penetration of Sn ion into the biofilm for Examples 4 (i.e., Sn+Taurate containing compositions) vs. the Comparative Examples 3 (i.e., containing Sn+SLS) and Example 6 (Sn+SLS+Betaine). More surprisingly the Inventive Example 5 (containing Sn+Taurate+Cocoamidopropyl betaine) has greater stannous penetration than that of Example 4 of Sn+Taurate only.

The surprising combination of Stannous and the combination of surfactants of Taurate and cocoamodipropyl betaine has a significant improvement in the bacterial endotoxin neutralization of the stannous ion penetration into the biofilm.

### Example 3: RCTA Barrier Disruption Test

Test Method 3 is conducted to measure barrier disruption of a formula. The higher numerical score the better, showing that the cells' barrier are more tolerant/less irritated and not impaired.

TABLE 4
RCTA Barrier Disruption test

| | % Cell Barrier | |
|---|---|---|
| Treatment | Deterioration at 2 hr | Grouping * |
| Example 2 | 0.9168 | B |
| Example 4 | 0.9315 | B |
| Example 5 | 0.9603 | A |
| ²Example 7 | 0.7772 | C |

* Same as above.
²Same as above.

The results in Table 4 demonstrate the significantly higher tolerance to the gingival barrier deterioration of the human gingival epithelial cells for the Inventive Examples 5 (containing Sn+Taurate+Cocamidopropyl Betaine) maintains the integrity of the cell barrier deterioration better than those formulation compositions containing Taurate surfactants Examples 4 and 2, and also the commercial comparative Example 7 (composition containing Zn+Taurate+Cocoamidopropyl Betaine).

The surprising combination of Stannous and the combination of surfactants Taurate and Cocoamidopropyl Betiane has a significant improvement in the bacterial endotoxin neutralization of the stannous ions penetration into the biofilm and reduces the gingival cell barrier deterioration of the gingival epithelium that if compromised can allow bacterial toxins to infiltrate and cause gingival inflammation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition comprising:
(a) 0.01% to 5%, by weight of the composition, of a stannous ion source;
(b) 0.1% to 5%, by weight of the composition, of a Taurate surfactant represented by formula 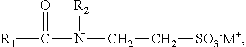
wherein R₁ is a saturated or unsaturated, straight, or branched alkyl chain with 6 to 18 C atoms; R₂ is H or methyl, and M is H, sodium, or potassium; and
(c) from 0.01% to 5%, by weight of the composition of an amphoteric surfactant.

2. The oral care composition of claim 1, wherein the R₁ is a saturated or unsaturated, straight, or branched alkyl chain with 8 to 18 C atoms, optionally wherein the taurate surfactant comprises one or more of potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate.

3. The oral care composition of claim 1, wherein the amphoteric surfactant comprises a betaine surfactant selected from cocoamidopropyl betaine, lauramidopropyl betaine, oleamidopropyl betaine, tallowamidopropyl betaine, cocamidopropyl hydroxysultaine, and combinations thereof.

4. The oral care composition of claim 1, wherein the betaine surfactant is present in the level of 0.02% to 4%, by weight of the composition.

5. The oral care composition of claim 1, wherein the oral care composition is substantially free of any insoluble zinc ion source.

6. The oral care composition of claim 1, wherein the stannous ion source is selected from stannous chloride, stannous fluoride, stannous acetate, stannous gluconate, stannous oxalate, stannous sulfate, stannous lactate, stannous tartrate, stannous iodide, stannous chlorofluoride, stannous hexafluorozirconate, stannous citrate, stannous malate, stannous glycinate, stannous carbonate, stannous phosphate, stannous pyrophosphate, stannous metaphosphate, and combinations thereof.

7. The oral care composition of claim 1, wherein the stannous ion source comprises stannous chloride, stannous fluoride or a combination thereof.

8. The oral care composition of claim 1, further comprising 0.5% to 5%, by weight of the composition, of a fluoride ion source selected from stannous fluoride, sodium fluoride, potassium fluoride, sodium monofluorophosphate, and mixtures thereof.

9. The oral care composition of claim 1, wherein the oral care composition comprises 10% to 60%, by weight of the composition, of total water content.

10. The oral care composition of claim 1, further comprising 0.01% to 5%, by weight of the composition, of a thickening system selected from a thickening polymer, a thickening silica, or a combination thereof.

11. The oral care composition of claim 1, further comprising 1% to 35%, by weight of the composition, of an abrasive selected from a calcium-containing abrasive, a silica abrasive, or a combination thereof.

12. The oral care composition of claim 1, wherein the composition is free of glycyrrhetinic acid and salts thereof.

13. The oral care composition of claim 1, wherein the composition has a pH of 4.0 to 9.0.

14. The oral care composition of claim 1, wherein the oral care composition is a single-phase toothpaste.

15. A method of preventing or mitigating plaque formation on tooth enamel comprising the step of brushing teeth with an oral care composition of claim 1.
